(19) United States
(12) Patent Application Publication
(10) Pub. No.: US 2002/0068339 A1
(43) Pub. Date: Jun. 6, 2002

(54) Microbead immobilization of enzymes

(76) Inventors: Henry A. Pfeffer, Hamilton, NJ (US); Catherine-Ann Cukras, St. Louis, MO (US); Robert K. Prud'homme, Princeton Junction, NJ (US)

Correspondence Address: Patent Administrator, FMC Corporation, 1735 Market Street, Philadelphia, PA, 19103, US

(21) Appl. No.: 09/909,003

(22) Filed: Jul. 19, 2001

Related U.S. Application Data
(63) Continuation-in-part of application No. 09/395,465, filed on Sep. 14, 1999.
(60) Provisional application No. 60/101,210, filed on Sep. 21, 1998.

(51) Int. Cl. C12N 11/10; C12N 11/00

(52) U.S. Cl. 435/178; 435/174

(57) ABSTRACT

Hydrocolloid gel beads containing enzymes immobilized therein are useful for catalyzing organic transformations in non-aqueous media. The enzyme is imbibed into a dehydrated hydrocolloid polymer gel bead. The resulting enzyme-laden bead may be dehydrated, if desired. A preferred hydrocolloid is carrageenan, especially kappa carrageenan. The resulting hydrocolloid gel beads are particularly adapted for catalyzing asymmetric transformations.

### CROSS-REFERENCE TO RELATED APPLICATIONS

This application is a continuation-in-part of U.S. application Ser. No. 09/395,465, filed Sep. 14, 1999, allowed Mar. 9, 2001, which claims priority on U.S. Provisional Application No. 60/101,210, filed Sep. 21, 1998.

### FIELD OF THE INVENTION

This invention is directed to physically immobilizing enzymes for use in non-aqueous enzymatic reactions. In particular, this invention relates to enzymes immobilized on dehydrated hydrocolloid polymer gel beads and to their use.

### BACKGROUND OF THE INVENTION

Enzymes are commercially attractive, high specificity catalysts for organic transformations. The productivity improvements potentially provided by biocatalysis include high local enzyme concentrations, recyclability, and increased stability. Because biocatalysts are highly specific catalysts, the desired product can be produced in high yield, eliminating waste streams and undesired byproducts.

Enzymes are generally useful in catalyzing only specific reactions, and generally are useful only at relatively low temperatures and pressures. In addition, most organic compounds of commercial interest are not soluble in water, and it is frequently difficult and expensive to recover the desired product from water. However, the potential advantages in carrying out organic transformations with enzymes outweigh these disadvantages. Thus, there have been attempts to develop enzymes that will function in non-aqueous solvents.

For commercial applications, the enzyme must be recovered so it can be reused. The recovery and reuse of enzymes is difficult. Using currently available techniques of enzyme recovery, either the method of removal contaminates the enzyme, rendering the enzyme unsuitable for reuse, or the activity of the enzyme is destroyed in the recovery process. In either case, removing the biofunctional enzyme from a reaction mixture that it catalyzes has proved difficult. In addition, when a dry enzyme is used in a non-aqueous solvent, the enzyme tends to agglomerate, reducing the surface area available to catalyze the reaction, thus reducing the rate of reaction.

Use of immobilized enzymes offers a potential solution to the problems of separation, recovery, and reuse. However, the enzyme must be immobilized without compromising its activity. Methods of enzyme immobilization include covalent binding, non-covalent binding, and physical entrapment. Immobilizing enzymes by covalently bonding them to a carrier prevents the enzyme from leaking from the carrier regardless of the stringency of the conditions. However, this form of immobilization generally alters the conformational structure and reactivity of the active site.

Non-covalent bonds such as hydrophobic binding, polar binding electrostatic interactions and hydrogen bridge binding (adsorption) have been used to associate the enzyme with a carrier material without forming covalent bonds. Because the binding is not as strong as covalent bonding, the conformation of the enzyme is usually not significantly altered and therefore the reactivity of the enzyme is not severely reduced. However, this weaker binding makes it possible for the enzyme to leak from the carrier more easily.

Entrapment of the enzyme does not involve any type of chemical binding, but only physically restricts the enzyme's movement within a polymer matrix. Therefore it does not interfere with the enzyme conformation. However, depending on the method of entrapment, the enzyme may either be damaged if the conditions are too stringent or simply be occluded so that its reactivity is reduced.

Therefore, a need exists for an improved method for immobilizing enzymes for use as catalysts in non-aqueous solvents. The method must prevent the enzyme from diffusing into the reaction medium, but must not significantly reduce its activity.

### SUMMARY OF THE INVENTION

In one aspect, this invention is a method for immobilizing an enzyme for use as a catalyst in non-aqueous solvents by imbibing the enzyme into dehydrated hydrocolloid polymer gel beads. More particularly, the gel beads are prepared by a process comprising:

(a) forming dehydrated gel beads, the gel beads having a network structure capable of swelling in aqueous media and an average particle size of about 5 microns to 150 microns in diameter; and

(b) imbibing an aqueous solution of the enzyme into the dehydrated hydrocolloid gel beads.

Optionally, the gel beads may be dehydrated after step (b). The enzyme may be an oxidoreductase, transferase, hydrolase, lyase, isomerase, ligase, decarboxylase, carboxylase, aldolase, thiolase, or synthase. In a preferred embodiment, the hydrocolloid is carrageenan, more preferably kappa carrageenan. A preferred method of dehydration is lyophilization.

In another aspect, the invention is hydrocolloid gel beads (sometimes referred to as “gel beads” or as “microbeads”) comprising a hydrocolloid and an enzymatically effective amount of an immobilized enzyme in the beads, in which the gel beads have an average particle size of about 5 microns to 150 microns in diameter.

In yet another aspect, the invention is the use of the hydrocolloid gel beads to carry out chemical transformations in non-aqueous solvents, such as chemical transformations that produce a chiral product.

The invention has direct application to enzymatic transformations in non-aqueous solvents, such as anhydrous or nearly anhydrous organic solvents. In particular, the invention is particularly useful for asymmetric transformations catalyzed by enzymes in non-aqueous solvents. In aqueous buffer, enzyme may diffuse out of the microbeads into the bulk solvent. Therefore the enzyme-containing gel beads may not be well suited for aqueous reaction systems in which enzyme recycling is desired.

### DETAILED DESCRIPTION OF THE INVENTION

In one aspect, the invention is a process for immobilizing enzymes, in which the enzyme is imbibed in a dehydrated bead of a bead-forming hydrocolloid polymer. In another aspect, the invention is the beads thus formed.

### Hydrocolloid Gel Beads

The bead-forming polymers suitable for use include various hydrocolloids that gel upon cooling. Carrageenan, preferably kappa carrageenan (κ-carrageenan), and other polymers that are capable of being formed into gel beads and which have a network structure capable of imbibing or entrapping enzymes are suitable for use in the invention. In addition, it is highly beneficial that the dehydrated beads are capable of swelling in the presence of aqueous solutions or suspensions of the enzyme to facilitate imbibition of the enzyme into the dried bead.

Other polymers that may be suitable include agars, agaroses, algins, low methoxyl pectins, gellans, furcellaran, curdlan, chitosan, konjac glucomannan and various derivatives thereof, and mixtures of two or more of the foregoing, as well as hydrocolloid mixtures such xanthan/locust bean gum, locust bean gum/agar, cassia/agar, cassia/xanthan, konjac/xanthan, carrageenan/locust bean gum, konjac/carrageenan, and konjac/starch.

Carrageenan is particularly desirable as the hydrocolloid polymer due to its networked structure, its ability to form very fine particle size beads, its ability to swell in aqueous media and its ability to interact with proteins.

κ-Carrageenan is a linear polysaccharide made up of alternating 1,3-linked B-D-galactose-4-sulfate and 1-4-linked 3,6-anhydro-α-D-galactose as shown in FIG. 1.

[chemical structure]

This polymer is believed to form a gel network in two steps. The first step involves the partial association of polymer chains into double helices. The association of helices into “domains ” by the addition of a cation (usually potassium) produces the gel network. The gelation temperatures of κ-carrageenan polymers are reported to be dependent on the cation concentration, and relatively independent of carrageenan concentration. For example, the most common method used to immobilize biological cell suspensions in carrageenen gels is to prepare a carrageenen solution in the absence of cations, and when the solution has cooled to about 45° C., the cell suspension is added and the gel is configured into the desired geometry. Once the gel has cooled, it is cured with a potassium chloride solution.

Use of a small diameter bead of 20 μm or less is believed to minimized diffusion resistance and thereby facilitate imbibition and immobilization of enzymes carried in aqueous media, as well as promote availability of the enzyme at the active site. However, beads having an average diameter of about 5 microns to about 150 microns, more preferably 5 microns to 50 microns may be used.

The polymer beads may be prepared by methods known in the art. Particularly desirable are the very fine gel beads formed according to the process described in Thomas, U.S. Pat. No. 5,662,840, in which a hydrocolloid sol, such as carrageenan, is intimately contacted with sufficient atomizing gas to immediately flash cool the sol to a temperature below the gelation temperature of the sol. This method has the advantage of producing very fine uniform gel beads having an average diameter of about 5 microns to 50 microns, preferably of about 10 microns up to about 20 microns.

Nearly all enzymes are suitable for use in this invention. Preferred enzymes include oxidoreductases (including but not limited to, dehydrogenases, oxidases, reductases, hydroxylases, monooxygenases, peroxidases, and nitrogenases), transferases (including but not limited to, proteases, esterases, aminotransferases, phosphatases, nucleases, phosphodiesterases, and phosphorlases), hydrolases, lyases (including but not limited to, aconitase, fumarase, enolases, crotonase, dehydrases, and aspartase), isomerases (including but not limited to racemases, epimerases, and mutases), and ligases (including but not limited to synthetases and carboligase). Additional preferred enzymes, but are not limited to, decarboxylases, carboxylases, aldolases, thiolases, and synthases. Hydrolases, for example lipases and proteases, are especially suitable enzymes for use in non-aqueous solvents.

The immobilization technique involves imbibing the enzyme into the pre-formed hydrocolloid beads. The beads may be manufactured using a carrageenan mixture comprising from about 0.5 to about 4 wt %, preferably about 2 wt % carrageenan, 0.05 to about 0.4 wt %, preferably about 0.2 wt % potassium chloride, 0.025 to about 0.2 wt %, preferably about 0.1 wt % calcium chloride, and 0.025 to about 0.2 wt %, preferably about 0.1 wt % sodium benzoate, in deionized water.

The beads are then dehydrated. The method of dehydration is not critical, and any suitable method may be used. A convenient and preferred method is lyophilization, which may be conducted on either a laboratory or large scale, using the techniques described generally in Methods in Enzymology, “Guide to Protein Purification”, 182, 77-8, Academic Press. Large-scale lyophilizations are useful in carrying out the present invention commercially and are well known to those skilled in the art. Regardless of scale, lyophilization involves the rapid freezing of the microbeads and subsequent removal of the water contained therein by sublimation under a vacuum. An alternative method of dehydration involves contacting the gel microbeads with a water miscible alcohol, e.g., ethanol or isopropyl alcohol.

Following dehydration, the enzyme is imbibed in the dehydrated beads from an enzyme solution. This imbibition step generally involves immersing or suspending the dehydrated beads in an aqueous solution of the enzyme, preferably with stirring or agitation, or spraying an aqueous solution of the enzyme onto the dehydrated beads, for a period of time sufficient to permit the beads to swell in the aqueous solution and to allow the entrained enzyme to bind to the pre-formed bead, generally for about 0.5 to about 8 hours. The amount of solution used for swelling and the enzyme concentration in the solution will vary depending on the polymer used for the bead and on the enzyme being used. In general the amount of aqueous enzyme solution used should be equal to or in excess of the moisture loss on lyophilization of the gel bead; it should be sufficient to restore the bead to a fully hydrated condition. Excess solution and excess enzyme, for example up to a 50% excess, is desirably used to maximize the amount of enzyme imbibed into the bead.

For the carrageenan beads used to illustrate this invention, the amount of solution used was 150% of the amount calculated to be enough to swell the gel beads to their original (pre-lyophilized) moisture content of about 2 wt %. The amount of enzyme used may be about 0.05 to about 0.5 g of enzyme per gram of hydrocolloid, preferably about 0.25 g of enzyme per gram of hydrocolloid (0.2 g per g of dried hydrocolloid). The aqueous solution may suitably contain from about 0.05 wt % to about 40 wt % enzyme, preferably about 0.05 wt % to about 5 wt %, more preferably about 0.05 wt %.

Depending on the enzyme, the aqueous solution of enzyme may also contain compatible water-soluble buffers and stabilizers, particularly for those enzymes whose activity and/or ability to bind to the gel bead is pH dependent. For example, for the enzyme subtilisin Carlsberg, it is advantageous to use a pH of about 7.8, advantageously a 20 mM potassium phosphate buffer adjusted to pH of 7.8 with potassium hydroxide.

It is well known that an increase in polarity of the enzyme can improve the catalytic activity of the enzyme in organic solvents. This increase in polarity may be accomplished by assuring that a small amount of water is in intimate contact with the enzyme. With subtilisin Carlsberg, for example, it is desirable to have water present at a level equal to about 10 μL/mg of enzyme in order to maximize the activity of this enzyme. In general, this amount of water is obtained by using an aqueous solution of the enzyme for imbibation of the enzyme, and can be altered by controlling the amount of dehydration or adding additional water to the non-aqueous reaction medium. Alternatively, the increase in polarity of the active site of the enzyme may be achieved by genetically engineering the enzyme to directly alter the polarity of the active site. In one embodiment of the present invention with subtilisin Carlsberg as the enzyme, water is present at about 10 μL/mg and the pH is adjusted to 7.8.

Following imbibition of the enzyme, the excess liquid is then removed from the beads by any suitable means, for example by centrifugation or decantation. The beads may then be recovered and used as such or further dehydrated and stored or shipped for later use. The beads may be lyophilized or otherwise dehydrated in the manner described above to provide a dehydrated gel bead in which a enzymatically effective amount of enzyme has been immobilized.

### Enzymatic Reactions

In another aspect, the invention is the use of the gel beads of the invention to carry out chemical transformations in non-aqueous solvents. In this specification, “substrate” and “reaction substrate” means any chemical entity, reactant, or reagent susceptible to a chemical transformation or transformations, including isomerism, by interaction with an enzyme. “Enzymatically effective amount” of enzyme means the amount of enzyme necessary to carry out a measurable chemical transformation on a substrate, i.e., measurable conversion of substrate to product.

The enzymatic reaction may be conveniently carried out by adding a mixture of enzyme-containing microbeads in a buffer appropriate for the enzyme to a mixture of the reaction substrate in suitable non-aqueous solvent. When the microbead-containing mixture is added, a two-phase mixture typically forms. The non-aqueous solvent is typically the continuous phase, and the microbead-containing buffer is the discontinuous phase. While not being bound by any theory or explanation, it is believed that the substrate diffuses from the non-aqueous phase to the phase containing the microbeads, and the reaction products diffuse back into the organic phase.

Suitable solvents are non-aqueous solvents and mixtures of aqueous solvents that are hydrophobic enough to prevent diffusion of the enzyme into the bulk solvent, but sufficiently polar to dissolve both the substrate and the reaction products. The microbeads containing the enzyme should be essentially insoluble in the non-aqueous solvent. Preferably, the non-aqueous solvent is essentially insoluble in water and water is essentially insoluble in the non-aqueous solvent so that the non-aqueous solvent does not dehydrate the microbeads and deactivate the enzyme. Non-aqueous solvents that are essentially anhydrous i.e., contain less than 0.01 wt % water, or contain from 0.01 wt % up to a few percent water may be used. Preferably the non-aqueous solvent contains less than 1 wt % water. To prevent dehydration of the microbeads, the non-aqueous solvent may be equilibrated with water prior to use.

Suitable solvents include, for example, organic liquids in which water has little or no solubility and mixtures thereof, such as, for example, hydrocarbons, alcohols, ethers, and esters. Suitable liquid alcohols include, for example, 1-butanol, 2-butanol, 1-pentanol, 3-methyl-3-pentanol, 1-hexanol, cyclohexanol, 1-heptanol, iso-octyl alcohol, 1-octanol, 1-nonanol, and 1-decanol. Other suitable organic solvents are mixtures of alcohols, preferably alcohols of one to four carbon atoms, with liquid hydrocarbons or liquid hydrocarbon mixtures, such as mixtures of 2-propanol with isooctane and/or n-octane. Other hydrocarbons that may be used include, for example n-pentane, 3-methylpentane, n-hexane, iso-hexane, cyclohexane, iso-heptane, n-heptane, and n-nonane. Mixtures of hydrocarbons, such as mixed pentanes, mixed hexanes, mixed heptanes, mixed octanes, ligroin, and petroleum ethers may also be used.

The ease with which the microbeads and the reaction products can be recovered from the non-aqueous solvent and the ease with which the solvent can be recovered and recycled, as well as cost, safety, and environmental considerations, may determine the choice of solvent. Lower boiling solvents, for example, are typically more readily removed from the reaction products than higher boiling solvents.

### Industrial Applicability

The enzyme-containing hydrocolloid gel microbeads of the invention offer significant advantages over aqueous enzyme systems for transformations in non-aqueous solvents. In particular, the gel beads of the invention are readily separated from the non-aqueous solvent. Water, however, forms a fine dispersion that cannot be easily separated from the non-aqueous solvent.

The enzyme-containing hydrocolloid gel microbeads can be used to promote practically any enzymatic transformation that can be carried out in a non-aqueous solvent, such as, hydrolysis of esters; transesterification; lactonization of esters of hydroxy acids; acylation of glycols, steroids, and sugars; addition of hydrogen cyanide to aldehydes; and hydroxylation of phenols. For example J. Deetz and D. Rozzell, Ann. New York Acad. Sci., 1987, 230-234, have shown that alcohol dehydrogenase (ADH) catalyzes the oxidation of alcohols and the reduction of carbonyl compounds such as aldehydes and ketones in a hexane/alcohol (10 mM cinnamyl alcohol, 10 mM octanol)/water (0.1%) solvent mixture.

Many transformations involving compounds of pharmacological interest are carried out in non-aqueous solvents, because most intermediates and products are not soluble in water. Because frequently only one isomer out of several possible stereoisomers is biologically or pharmacologically useful, one particularly desirable application for the use of enzymes in non-aqueous solvents is the catalysis of stereospecific reactions. Hydrolases, for example lipases and proteases, are especially suitable enzymes for as catalysts for asymmetric transformations in non-aqueous solvents.

Asymmetric transformation refers to a reaction that produces a chiral product. The term includes stereoselective and stereospecific reactions, as well as regioselective reactions. As is well known, asymmetric molecules have at least two optical isomers. A stereoselective synthesis produces one optical isomer, or diasteroisomer, in preponderance over the other possible optical isomers. Optical isomerism is discussed in Stereochemistry of Carbon Compounds, E. L. Eliel, McGraw-Hill, New York, 1962. Enzymatic production of optically active compounds in aqueous solution is discussed on pages 75-78.

Chiral product, as used herein, refers to a product that contains an excess of one optical isomer. The chiral product may be produced from a chiral substrate or from an achiral substrate. Preferably, the asymmetric transformation produces a product in which there is at least a 10% molar excess of one optical isomer, more preferably at least a 50% molar excess of one optical isomer. With the use of the immobilized enzymes of the invention, asymmetric transformation that produce more than 90%, preferably substantially all, of the desired optical isomer should be possible.

The field of asymmetric transformations catalyzed by enzymes in non-aqueous solvents, to which the present invention is applicable, has been reviewed by A. Klibanov, Acc. Chem. Res. (1990), 23, 114-120. Most applications involve hydrolases, namely, lipases and proteases. Lipases and proteases catalyze the hydrolysis of chiral esters. This allows the transesterification of racemic alcohols. For example, pig liver carboxyl esterase and yeast lipase have been used to produce primary and secondary chiral alcohols (see, for example, B. Cambou, B. and A. M. Klibanov, J. Am. Chem. Soc. (1984) 106, 2687-2692.

The invention is also applicable to the resolution of racemic alcohols into chiral alcohols by the lipase-catalyzed acylation of carboxylic acids as described, for example, by G. Langrand, M. Secchi, G. Buono, J. Baratti, and C. Triantaphylides, in Tetrahedron Lett. (1985) 26, 1857-1860, reviewing the preparation of optically active carboxylic acids and esters, stereoselective production of lactones via lipases, the regioselective acylation of glycols, steroids, and sugars. Non-hydrolase based conversions that may be effected in accordance with the invention, such as the addition of hydrogen cyanide to aldehydes catalyzed by the enzyme mandelonitrile lyase, are disclosed in R. X. Effenberger and A. M. Klibanov, J. Am. Chem. Soc, (1985) 107, 5448-5450.

The advantageous properties of this invention can be observed by reference to the following examples, which illustrate but do not limit the invention. In the specification, examples, and claims, unless the context indicates otherwise, all parts and percentages are by weight and temperatures are in Celsius (°C.), and all pressures are in pounds per sq. in. (psi).

### EXAMPLES

### Example 1

Transesterification of N-Acetyl-L-phenylalanine Ethyl Ester with Propanol Using Subtilisin Carlsberg Enzyme

A mixture was prepared containing 0.0285 g (0.000121 mole) of N-acetyl-L-phenylalanine ethyl ester, 0.0164 g (0.000061 mole) of nonadecane, and 0.75 mL of propanol in 9.25 mL of octane. A 2 mL aliquot of this solution was placed in a 5 mL vial. To the vial were added 0.0012 g of subtilisin Carlsberg enzyme powder and 12 μL of water. This mixture was shaken at a temperature of 40-42° C. Periodically, small samples were removed and analyzed by gas chromatography. After 60 minutes, analysis indicated that 0.00486 g of N-acetyl-L-phenylalanine propyl ester had been formed. Prior to running this reaction the subtilisin Carlsberg enzyme powder had been dissolved in 20 mM potassium phosphate buffer solution at a concentration of 5 mg/mL. After adjusting the pH to 7.8 with potassium hydroxide, the solution was lyophilized by being frozen in liquid nitrogen and then putting the sample under a vacuum of 2.67 Pa at −45° C. for 30 hours.

### Example 2

Preparation of Carrageenan Gel Beads Containing Embedded Subtilisin Carlsberg Enzyme

Carrageenan gel beads containing subtilisin Carlsberg enzyme were prepared by the procedure described in Thomas, U.S. Pat. No. 5,662,840. A solution containing 2.5 wt % carrageenan, 0.2 wt % potassium chloride, 0.1 wt % calcium chloride, and 0.1 wt % sodium benzoate in deionized water was prepared. The resulting solution was heated and maintained at a temperature of about 92-93° C. with stirring to prevent gelation. This solution was then pumped through heated tubing to a high-pressure spray head at a flow rate of 24 mL/min. Just prior to reaching the spray head, a cold solution of 25 mg of subtilisin Carlsberg enzyme/mL of 20 mM potassium phosphate buffer, the pH of which had been adjusted to 7.8 with potassium hydroxide, was injected into the carrageenan solution at a rate of 6 mL/min. This created a solution containing 2 wt % carrageenan and 5 mg of subtilisin Carlsberg/mL. Simultaneously, a stream of air was passed through the spray head at a pressure of 51.7 kPa (75 psi), impinging on the carrageenan/enzyme solution as it left the spray head and atomizing the aqueous solution. The pressure differential instantly dispersed the carrageenan/enzyme solution into droplets, which immediately cooled and solidified into beads having an average diameter of about 20 microns. The resulting carrageenan beads containing subtilisin Carlsberg enzyme were then lyophilized and stored under anhydrous conditions for later use. Calculations determined that 5 mg of enzyme were contained in 25 mg of dried beads.

### Example 3

Transesterification of N-Acetyl-L-phenylalanine Ethyl Ester with Propanol Using Subtilisin Carlsberg Enzyme Embedded in Carrageenan Beads

A mixture was prepared containing 0.0280 g (0.000119 mole) of N-acetyl-L-phenylalanine ethyl ester, 0.0145 g (0.000061 mole) of nonadecane, and 0.75 mL of propanol in 9.25 mL of octane. A 2 mL aliquot of this solution was placed in a 5 mL vial. To 5 mg of the lyophilized beads produced in Example 2 was added 250 μL of water to rehydrate the beads to their condition before lyophilization. These rehydrated beads were then added to the reaction vial, which was shaken at 40° C. Periodically, small samples of the reaction mixture were removed and analyzed by gas chromatography. After 60 minutes, analysis indicated that 0.00199 g of N-acetyl-L-phenylalanine propyl ester had been formed. Carrageenan beads containing subtilisin Carlsberg enzyme have been shown to absorb 25% of the N-acetyl-L-phenylalanine ethyl ester, reducing the amount of this starting material that was available for transesterification in the original mixture to 0.0042 g.

### Example 4

Preparation of Subtilisin Enzyme Solution

A 20 mM potassium phosphate buffer solution was prepared and adjusted to a pH of 7.8 with potassium hydroxide. To 55.5 mL of the buffered solution was added 0.2767 g of subtilisin Carlsberg enzyme powder. The resulting solution contained 0.49 wt % of the enzyme. This enzyme solution was stirred in a refrigerator for 30 min prior to being used. For longer term storage the solution was lyophilized as described in Example 1 and then reconstituted for use as needed.

### Example 5

Preparation of Carrageenan Beads Containing Imbibed Subtilisin Carlsberg Enzyme

Carrageenan beads having a content of 2 wt % carrageenan, but no enzyme, were made according to the process of Example 2. These beads were lyophilized in the manner described in Example 1. Beads weighing 0.7515 g were added to 55.5 mL of the enzyme solution prepared in Example 4, and the mixture was stirred in a refrigerator for two hours. At the conclusion of this period, the mixture was transferred to a centrifuge tube in which it was centrifuged at 4000 rpm at 10° C. for 15 min. During centrifugation, the carrageenan formed a sticky pellet, allowing 30.7 mL of supernatant liquid to be decanted. The carrageenan pellet was transferred to a freeze-drying bottle in which it was lyophilized. The supernatant liquid was analyzed by ultraviolet spectrophotometry at 280 nm according to the method of Pantoliano et al. (Biochemistry, 28, 7205 (1989)). The enzyme concentration was found to be 0.46 wt %. From the volume of supernatant, the solution imbibed by the dried carrageenan beads had been sufficient to restore the beads to a 3 wt % carrageenan gel containing 0.173 g of enzyme per gram of carrageenan. These beads were then lyophilized as described above and stored for later use.

### Example 6

Transesterification of N-Acetyl-L-phenylalanine Ethyl Ester with Propanol Using Subtilisin Carlsberg Enzyme Imbibed in Carrageenan Beads

A mixture was prepared containing 0.0282 g (0.000120 mole) of N-acetyl-L-phenylalanine ethyl ester, 0.0170 g (0.000063 mole) of nonadecane, and 0.75 mL of propanol in 9.25 mL of octane. A 2 mL aliquot of this solution was placed in a 5 mL vial. To 5.8 mg of the lyophilized beads produced in Example 5 was added 193 μL of water to rehydrate the beads to their condition before lyophilization. These rehydrated beads were then added to the reaction vial, which was shaken at 40° C. Periodically, small samples of the reaction mixture were removed and analyzed by gas chromatography. After 1 hr, analysis indicated that 0.00268 g of N-acetyl-L-phenylalanine propyl ester had been formed. Carrageenan beads containing subtilisin Carlsberg enzyme have been shown to absorb 25% of the N-acetyl-L-phenylalanine ethyl ester, reducing the amount of this starting material that was available for transesterification in the original mixture to 0.00423 g.

### Example 7

Preparation of Reagents

The following reagents were used in Examples 8-12.

Buffer Solution—A 10 mM bis-tris propane(1,3-bis[tris(hydroxymethyl)methyl-amino]propane) buffer solution was prepared and adjusted to pH 7.8 using hydrochloric acid.

α-Chymotripsin Solution—α-Chymotripsin (15.5 mg) was added to 15.5 mL of the buffer solution to produce a solution that contained 1 mg/mL of α-chymotripsin. α-Chymotripsin is a 25 kDa protease that specifically hydrolyzes peptide bonds adjacent to aromatic amino acid residues. The solution was shaken in a vortex mixer and stored in an ice water bath for 15 min prior to incorporation into microbeads.

α-Chymotripsin Containing Microbeads—The α-chymotripsin solution (5.55 mL) was added to 49.95 mL of the buffer solution to produce a solution that contained 0.1 mg/mL of a-chymotripsin. To this solution was added 0.715 g of lyophilized carrageenan microbeads that had been produced by a spray method and lyophilized in accordance with the present invention. The resulting mixture was stirred in an ice water in bath for 45 min.

N-succinyl-ala-ala-pro-phe-p-nitroanilide (NSNA) Solution—NSNA (59.1 mg) was added to 5.91 mL of N,N-dimethylformamide. The resulting mixture was mixed on a vortex mixer for 30 sec and cooled in an ice water bath.

### Example 8

Cleavage of the Peptide Bond Between Phenylalanine and p-Nitroaniline NSNA Using α-Chymotripsin in Buffer

A mixture of 14.5 mg of N-succinyl-ala-ala-pro-phe-p-nitroanilide (NSNA) and 1.45 mL of N,N-dimethylformamide was cooled in an ice water bath. To 14.7 mL of buffer solution was added (1) 150 μL of the NSNA solution and (2) 150 μL of a mixture of the α-chymotripsin solution with 10 mM of buffer whose enzyme concentration is 10 μg/mL. As noted above, these materials were prepared as described in Example 7.

α-Chymotripsin selectively cleaves the peptide bond between the phenylalanine and p-nitroaniline residues releasing p-nitroanaline, which absorbs at 410 nm. The reaction was monitored by spectrophotometrically following the increase in absorption at 410 nm. After 24 hr, the maximum absorption was reached and a final absorbance measurement taken. Enzyme activity was measured by the time necessary for the absorbance to reach 5% of the maximum absorbance. The results for 4 replicate samples: are 4.3 min, 4.0 min, 1.1 min, and 5.8 min.

### Example 9

Cleavage of the Peptide Bond Between Phenylalanine and p-Nitroaniline in NSNA Using α-Chymotripsin in Carrageenan Microbeads in Aqueous Solution

A mixture of 14.5 mg of NSNA and 1.45 mL of N,N-dimethy formamide was cooled in an ice water bath. To 14.7 mL of buffer solution was added (1) 150 μL of the NSNA solution and (2) 150 μL of the mixture of α-chymotripsin containing the carrageenan microbead mixture of Example 7 with 10 mM of buffer. The total enzyme concentration of the mixture was 10 μg/mL.

The time necessary for the absorbance to reach 5% of the maximum absorbance was measured as in Example 9. The results for 4 replicate samples are: 3.4 min, 1.6 min, 2.2 min, and 2.0 min.

### Example 10

Cleavage of the Peptide Bond Between Phenylalanine and p-Nitroaniline in NSNA Using α-Chymotripsin in Carrageenan Microbeads in a 1-Octanol Reaction Medium

The NSNA solution prepared in Example 7 (100 μL) was added to 8 mL of 1-octanol. The lyophilized carrageenan microbead mixture prepared in Example 7 (100 μL) was added to the mixture, producing a two-phase reaction medium. The solution became yellow, indicating that a reaction had occurred.

### Example 11

Cleavage of the Peptide Bond Between Phenylalanine and p-Nitroaniline in NSNA Using α-Chymotripsin in Carrageenan Microbeads in a 2-Propanol/Isooctane Reaction Medium

A reaction medium was prepared by mixing 2.0 mL of 2-propanol with 8.0 mL of isooctane. The NSNA solution prepared in Example 7 (100 μL) was added to 8 mL of the reaction medium. The lyophilized carrageenan microbead mixture prepared in Example 7 (100 μL) was added to the mixture, producing a two-phase reaction medium. The solution became yellow, indicating that a reaction had occurred.

### Example 12

Recovery and Reuse of the Microbeads

Five samples, each containing 6.0 mL of 1-octanol and 100 μL of the NSNA solution prepared in Example 7, were prepared. The lyophilized carrageenan microbead mixture prepared in Example 7 (200 μL) was added to the first sample, producing a two-phase reaction mixture, with small water/bead bubbles in the continuous organic phase. The mixture was shaken for 2 min, producing a yellow color that was monitored by spectrophotometrically following the increase in absorption at 410 nm. The reaction phases were allowed to separate, and the 1-octanol phase was removed with a pipette.

The second sample, third sample, fourth sample, fifth sample, and sixth sample were each added in order to the lyophilized carrageenan microbead mixture, and the process repeated with each sample. Reaction took place with the second sample, third sample, fourth sample, and fifth sample. No reaction occurred with the sixth sample.

Although the invention has been particularly shown and described with reference to the certain embodiments, those skilled in the art will appreciate that various modifications and changes in form and details may be made without departing from the spirit and scope of the invention. Having described the invention, we now claim the following and their equivalents.

What is claimed is:

1. Gel beads comprising a hydrocolloid and an enzymatically effective amount of an immobilized enzyme in the beads, in which the gel beads have an average particle size of about 5 microns to 150 microns in diameter.

2. The gel beads of claim 1 in which the hydrocolloid is carrageenan.

3. The gel beads of claim 2 in which the gel beads have a diameter of 5 microns to 50 microns.

4. The gel beads of claim 3 in which the enzyme is selected from the group consisting of lipases and proteases.

5. The gel beads of claim 1 in which the hydrocolloid is kappa carrageenan.

6. The gel beads of claim 1 in which the enzyme is selected from the group consisting of oxidoreductases, transferases, hydrolases, lyases, isomerases, ligases, decarboxylases, carboxylases, aldolases, thiolases, and synthases.

7. The gel beads of claim 1 in which the enzyme is selected from the group consisting of lipases and proteases.

8. The gel beads of claim 1 in which the gel beads have a diameter of 5 microns to 50 microns.

9. A method for carrying out a chemical transformation, the method comprising contacting a reaction substrate and gel beads in the presence of a non-aqueous solvent for a time sufficient to convert at least a portion of the substrate to a product, in which:
the gel beads comprise a hydrocolloid and an enzymatically effective amount of an immobilized enzyme;
the gel beads have a network structure capable of swelling in aqueous media and an average particle size of about 5 microns to 150 microns in diameter; and
the gel beads are substantially insoluble in the non-aqueous solvent.

10. The method of claim 9 in which the hydrocolloid is carrageenan.

11. The method of claim 10 in which the gel beads have a diameter of 5 microns to 50 microns.

12. The method of claim 11 in which the enzyme is selected from the group consisting of lipases and proteases.

13. The method of claim 12 in which the product is a chiral material.

14. The method of claim 12 in which the hydrocolloid is kappa carrageenan.

15. The method of claim 9 in which the hydrocolloid is kappa carrageenan.

16. The method of claim 9 in which the enzyme is selected from the group consisting of oxidoreductases, transferases, hydrolases, lyases, isomerases, ligases, decarboxylases, carboxylases, aldolases, thiolases, and synthases.

17. The method of claim 9 in which the enzyme is selected from the group consisting of lipases and proteases.

18. The method of claim 9 in which the gel beads have a diameter of 5 microns to 50 microns.

19. The method of claim 9 in which the product is a chiral material.

20. Gel beads comprising a hydrocolloid and an enzymatically effective amount of an immobilized enzyme,
the gel beads prepared by a process comprising::
(a) forming dehydrated gel beads, the gel beads having a network structure capable of swelling in aqueous media and an average particle size of about 5 microns to 150 microns in diameter; and
(b) imbibing into the dehydrated hydrocolloid gel beads an aqueous solution of the enzyme.

21. The gel beads of claim 20 in which the hydrocolloid is carrageenan.

22. The gel beads of claim 20 in which the hydrocolloid is kappa carrageenan.

23. The gel beads of claim 20 in which the enzyme is selected from the group consisting of oxidoreductases, transferases, hydrolases, lyases, isomerases, ligases, decarboxylases, carboxylases, aldolases, thiolases, and synthases.

24. The gel beads of claim 20 in which the enzyme is selected from the group consisting of lipases and proteases.

25. The gel beads of claim 20 in which the method additionally comprises, after step (b), the step of dehydrating the imbibed gel beads or the step of removing excess liquid from the imbibed gel beads.

26. The gel beads of claim 20 in which:
the hydrocolloid is carrageenan;
the gel beads have a diameter of 5 microns to 50 microns; and
the aqueous solution of enzyme contains about 0.05 to 40 wt % enzyme.

27. A method for carrying out a chemical transformation, the method comprising contacting a reaction substrate and gel beads in the presence of a non-aqueous solvent for a time sufficient to convert at least a portion of the substrate to a product,
in which:
the gel beads comprise a hydrocolloid and an enzymatically effective amount of an immobilized enzyme;
the gel beads have an average particle size of about 5 microns to 150 microns in diameter;
the gel beads are substantially insoluble in the non-aqueous solvent; and
the gel beads are prepared by a process comprising:
(a) forming dehydrated gel beads, the gel beads having an average particle size of about 5 microns to 150 microns in diameter; and
(b) imbibing into the dehydrated hydrocolloid gel beads an aqueous solution of the enzyme.

28. The method of claim 27 in which the hydrocolloid is carrageenan.

29. The method of claim 27 in which the hydrocolloid is kappa carrageenan.

30. The method of claim 27 in which the enzyme is selected from the group consisting of oxidoreductases, transferases, hydrolases, lyases, isomerases, ligases, decarboxylases, carboxylases, aldolases, thiolases, and synthases.

31. The method of claim 27 in which the enzyme is selected from the group consisting of lipases and proteases.

32. The method of claim 27 in which the method additionally comprises, after step (b), the step of dehydrating the imbibed gel beads or the step of removing excess liquid from the imbibed gel beads.

33. The method of claim 27 in which:
the hydrocolloid is carrageenan;
the gel beads have a diameter of 5 microns to 50 microns; and
the aqueous solution of enzyme contains about 0.05 to 40 wt % enzyme.

34. The method of claim 33 in which the product is a chiral material.
